(19) United States
(12) Patent Application Publication
(10) Pub. No.: US 2011/0070228 A1
(43) Pub. Date: Mar. 24, 2011

(54) METHOD AND APPARATUS FOR AUTOMATED CELL TRANSFER THERAPY AND HAIR TRANSPLANTATION

(75) Inventor: Allan Y. Wu, Cathedral City, CA (US)

(73) Assignee: SURGIMED BIOSCIENCES, INC., Cathedral City, CA (US)

(21) Appl. No.: 12/881,498

(22) Filed: Sep. 14, 2010

Related U.S. Application Data
(60) Provisional application No. 61/276,966, filed on Sep. 19, 2009.

(51) Int. Cl. A61K 39/395; A61P 17/14; A61N 2/00

(52) U.S. Cl. 424/133.1; 600/9

(57) ABSTRACT

Methods and apparatuses that selectively capture therapeutic cells associated with hair growth or development and their delivery to regions of hair loss. Cell capture and delivery is accomplished in part through the use of antibodies having binding sites specific to cell surface moieties characteristic of the therapeutic cells and having magnetic or paramagnetic features, which facilitates their immobilization to magnetizable rods that can be used to pierce the skin of the subject being treated for hair loss and the subsequent release of therapeutic cells to targeted sites beneath the epidermis.

### CROSS REFERENCE TO RELATED APPLICATIONS

This invention claims benefit of priority to U.S. patent application Ser. No. 61/276,966 filed on Sep. 19, 2009; the contents of which are herein incorporated by reference in their entirety.

### FIELD OF THE INVENTION

The present invention relates generally to a cell transfer therapy for the treatment of hair disorders, and more specifically to a method and apparatus for automated cell transfer therapy for hair transplantation.

### BACKGROUND OF THE INVENTION

A variety of approaches have been developed to address hair loss and baldness. Some approaches involve applying various medications to the scalp, some involve administering oral medications, while others involve surgical methods. One such approach, referred to as scalp reduction, involves the surgical removal of excess scalp to diminish the exposure of bare skin. Scalp reductions cannot be performed on all patients. For example, the degree of success in some patients is limited to the position of the exposed scalp, its surface area and the availability of nearby patches of hair.

Still another approach is referred to as hair transplantation. Hair transplantation is a process whereby hair follicles are surgically removed from hair follicle rich regions of the scalp, processed and then transplanted to depleted areas to give the appearance of hair restoration. This method is more labor intensive than scalp reduction and requires a hair graft technician, a surgeon and considerable time for graft preparation and final transfer. For example, an autologous hair graft transplantation procedure requires removal of hair bearing skin and meticulously dissecting out each follicle base. These grafts are then tediously transplanted by first starting a burr entry usually with a hollow syringe or instrument followed by painstakingly inserting the graft into the hole. The site is then allowed to heal. In addition, this extremely time consuming, labor intensive process requires considerable anesthesia/analgesia exposure times.

Improvements to hair transplantation procedures have been proposed to shorten the duration of the procedure. For example U.S. Pat. No. 5,643,308 issued to Markman provides a device and method that presses a series or array of dilators into the scalp. An enlarged probe portion expands the surrounding tissue and the array of dilators is removed thereby forming a plurality of cavities for inserting the hair follicle. Although Markman appears to decrease the time in forming cavities, Markman suffers from a common problem encountered in hair transplantation procedures. That is, conventional hair transplant procedures are limited by the number of follicle grafts obtained from the donor site. This limitation creates a dilemma for the surgeon in terms of deciding at times which regions must remain untreated if the number of donor grafts is in scant supply. In other words, since the approach is to transplant an entire hair follicle the procedure itself is inherently limited to the number of available or successfully collected hair follicles.

Hair follicles themselves are made up in part of cells. There is a growing body of evidence that hair stem cells or progenitor cells may be identified using specific cell surface moieties. Although these cells are believed to have identifiable markers, they have not been successfully exploited in transplantation techniques. Further, the difficulty of their capture and delivery leads to difficulties in exploiting a cell-based therapy.

Magnetic and paramagnetic antibodies have been used in laboratories and in clinical settings to label, manipulate or obtain specific cell types from a heterogeneous population of cells. These special antibodies have been used for various clinical therapy and diagnostic applications with great efficacy and safety. While demonstrated useful in some medical applications to date they have not been successfully demonstrated in procedures directed towards hair transplantation.

Accordingly, there remains a need to develop new devices and methods that provide improved hair transplantation procedures which require less time to perform and reduce the dependency on the number of available hair follicles.

### SUMMARY OF THE INVENTION

The present invention addresses the need to provide improved hair transplantation procedures and provides related benefits. This is accomplished through the development of apparatuses and methods that selectively capture therapeutic cells associated with hair growth or development and their selective delivery to regions of hair loss. Cell capture and delivery is accomplished in part through the use of antibodies having binding sites specific to cell surface moieties characteristic of the therapeutic cells and having magnetic or paramagnetic features, which facilitates their immobilization.

In a first aspect of the invention a method for the treatment of hair loss is provided, which includes excising donor tissue including therapeutic cells; treating the donor tissue to dissociate the therapeutic cells from the donor tissue; exposing the dissociated therapeutic cells to magnetically-sensitive antibodies capable of binding to a specific epitope of the therapeutic cells to form cell-antibody complexes; immobilizing the cell-antibody complexes to an electronically magnetizable rod; introducing the rod into a target site; releasing the cell-antibody complexes from the rod into the target site; and withdrawing the rod from the target site. The method may also include sequentially coating the rod with cell-antibody complexes of different cell types and in different layers to form a 3 dimensional graft prior to introducing the rod into the target site. In a related embodiment, each rod is initially coated with a layer of keratinocyte stem cells or stem cells then coated with a layer of dermal papilla cells to form a 3 dimensional structure for implantation into the target site.

The donor tissue and target site may be autologous, allogeneic or xenogeneic. The donor tissue is preferably skin tissue and includes at least one hair follicle. The donor tissue includes therapeutic cells which may include hair follicle cells, stem cells and progenitor cells. When the donor tissue includes adipose tissue, the therapeutic cells may be adipose-derived stem cells. The therapeutic cells may be used without genetic manipulation or may be genetically manipulated to enhance their function or to delivery gene therapy.

Magnetically-sensitive antibodies may be ferromagnetic or paramagnetic. The immobilization of such can be performed by magnetically polarizing a rod in the vicinity of the antibodies. Release or repulsion of the magnetically-sensitive antibodies and thus therapeutic cells may be accomplished by magnetically depolarizing or reversing the polarity of the rod. Magnetically-sensitive small molecules or drugs may also be immobilized and subsequently introduced together with the therapeutic cells.

In a related embodiment, a method for the treatment of hair loss is provided, which includes excising donor tissue comprising hair follicles from a subject; treating the donor tissue to dissociate therapeutic cells from the hair follicles; exposing the dissociated therapeutic cells to magnetically-sensitive antibodies capable of binding to a specific epitope of the therapeutic cells to form cell-antibody complexes; immobilizing the cell-antibody complexes to an electronically magnetizable rod; introducing the rod into a target site requiring hair growth; releasing the cell-antibody complexes from the rod into the target site; and withdrawing the rod from the target site. In some embodiments, the specific epitope comprises Nestin. In some embodiments, the target site is a region undergoing or to undergo a hair transplantation procedure.

In a related aspect an apparatus for use in the treatment of hair loss is provided, which includes a plurality of rods connected to a base and electronic circuitry. Each rod includes a magnetically shielded shaft and an electronically magnetizable tip. The electronic circuitry adjusts the magnetic field strength and polarity of the plurality of magnetic tips. Preferably, the number and pattern of rods is adjustable to simulate different hair densities and patterns. In some embodiments a retractable sheath covering the rod tip is provided to prevent damage to a cell-antibody complex immobilized thereon during penetration of skin tissue. In still further embodiments, the apparatus includes a handle connected to the base, which itself includes a button operably connected to the circuitry to adjust the magnetic field strength and/or polarity of the plurality of rods.

In another related aspect, an apparatus for use in the treatment of hair loss is provided, which includes a plurality of rods connected to a base, each rod including a magnetically shielded shaft and an electronically magnetizable tip. The plurality of rods includes a solid-state, predetermined magnetic polarity and strength without requiring electronic circuitry.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 depicts an apparatus 10 having a magnetizable rod 12 and a base 14, the magnetizable rod 12 being shown in a magnetized state.

FIG. 2 depicts a cell 16 immobilized to the tip of the magnetized rod 12 depicted in FIG. 1.

FIG. 3 represents an enlarged view of immobilizing a therapeutic cell 16, which is performed by magnetically interacting the tip of the rod 12 with a magnetically sensitive antibody 20, which itself is bound to the therapeutic cell 16.

FIG. 4 demonstrates piercing the tip of the rod 12 through the skin 22 to access the target site 26.

FIG. 5 depicts depolarizing the tip of the rod 12 to release the therapeutic cell 16 at the target site 26.

FIG. 6 depicts removing the rod 12 from the skin 22 thereby leaving the therapeutic cell 16 at the target site 26.

FIG. 7 depicts piercing the skin 22 with the apparatus 10 including an optional sheath 28 to protect the tip of the rod 12 and therapeutic cell 16 during the piercing step.

FIG. 8 depicts retracting the optional sheath 28 depicted in FIG. 7.

FIG. 9 depicts depolarizing the rod depicted in FIG. 7 to release the therapeutic cell 16 at the target site 26.

FIG. 10 is a second configuration of an apparatus 100 including a plurality of rods 112 attached to a base 114, which also provides a handle 116 with grip 118 and button 120.

### DETAILED DESCRIPTION OF THE INVENTION

The object of the invention is to address deficiencies in current methods and apparatuses used in the treatment of hair loss or hair replacement. As non-limiting examples, the invention may be used to treat androgenic alopecia, also referred to as male-pattern baldness, female-pattern baldness and the like. The skilled artisan will appreciate that while in preferred embodiments the hair loss subject is a human, any mammalian in need of hair restoration may be treated.

It is another object of the invention to provide a treatment method that is not limited to transplantation of an entire hair follicle at each target site. This is accomplished through selectively immobilizing therapeutic cells obtained from regions of higher hair density and transplanting the therapeutic cells in regions of lower hair density. Accordingly a single hair follicle may produce cells for implantation to many target sites within regions of lower hair density.

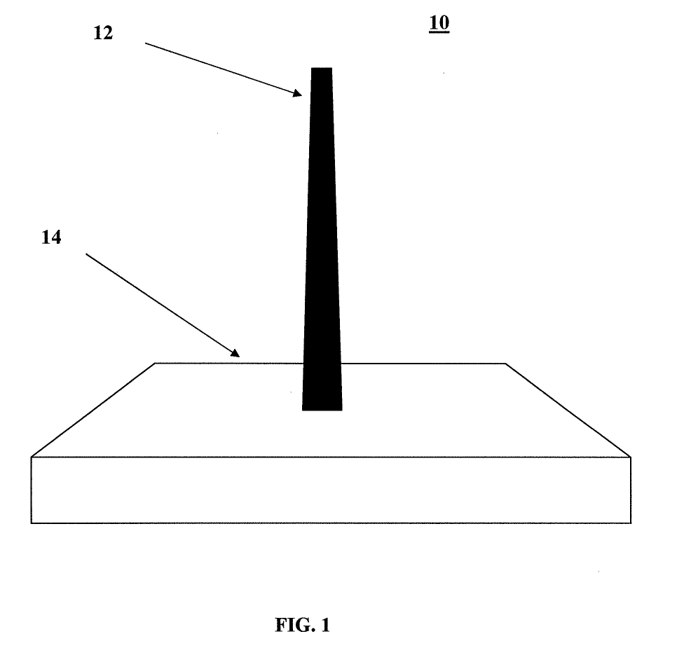

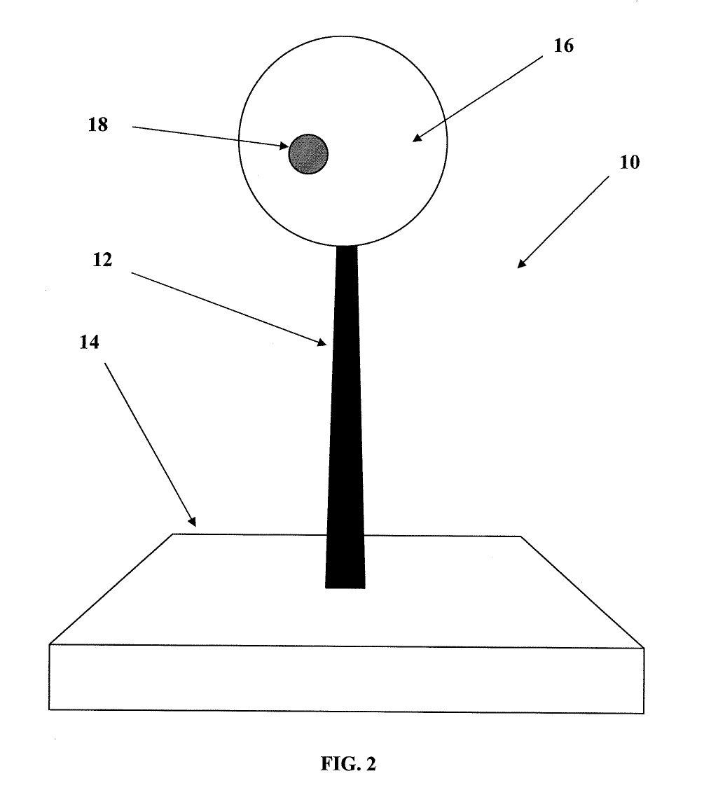

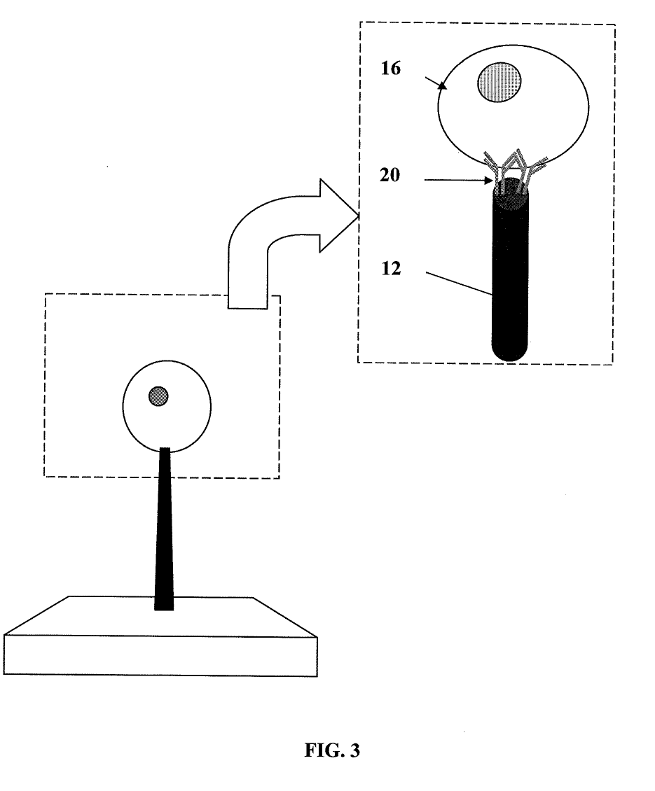

The above objects are accomplished by providing the apparatuses and methods overviewed in FIGS. 1-9. Referring to FIG. 1, an exemplary apparatus 10 is provided, which includes a magnetized rod 12 attached to a base 14. Turning to FIG. 2, exposing the magnetized rod 12 to a population of cells results in the immobilization of a therapeutic cell 16. Immobilization is depicted in more detail in FIG. 3, which demonstrates a magnetically-sensitive antibody 20 specifically bound to the therapeutic cell 16 thereby forming a cell-antibody complex. The cell-antibody complex is immobilized by magnetic interactions between the magnetically-sensitive antibody 20 and the magnetized rod 12.

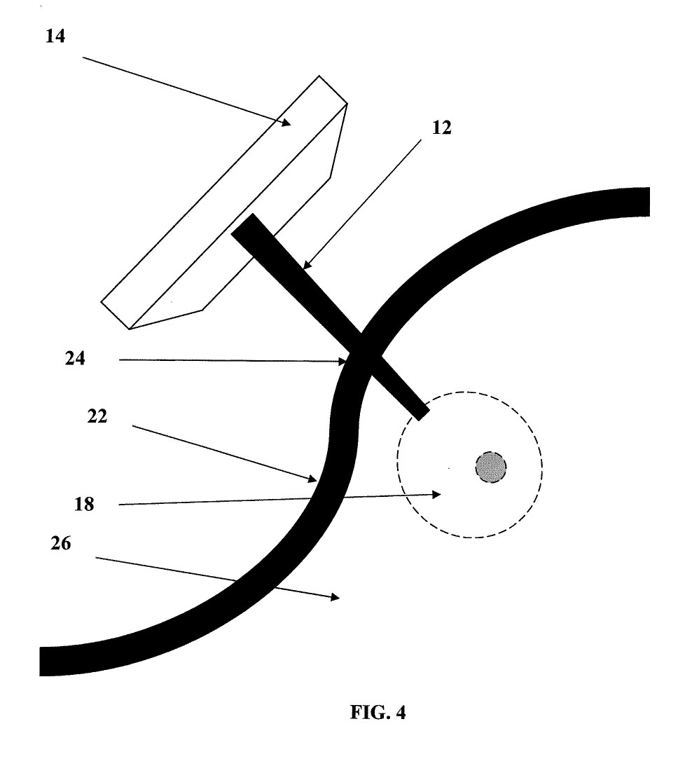

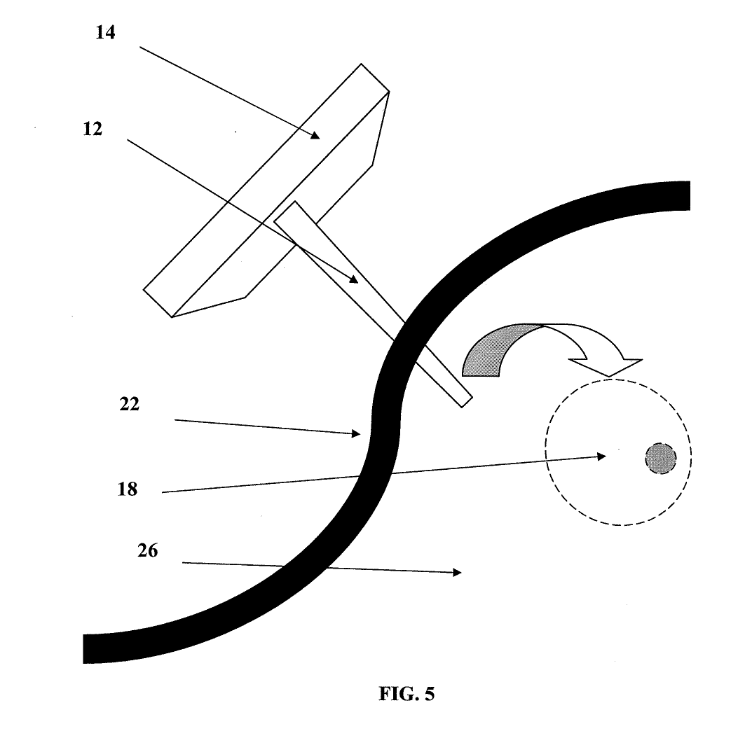

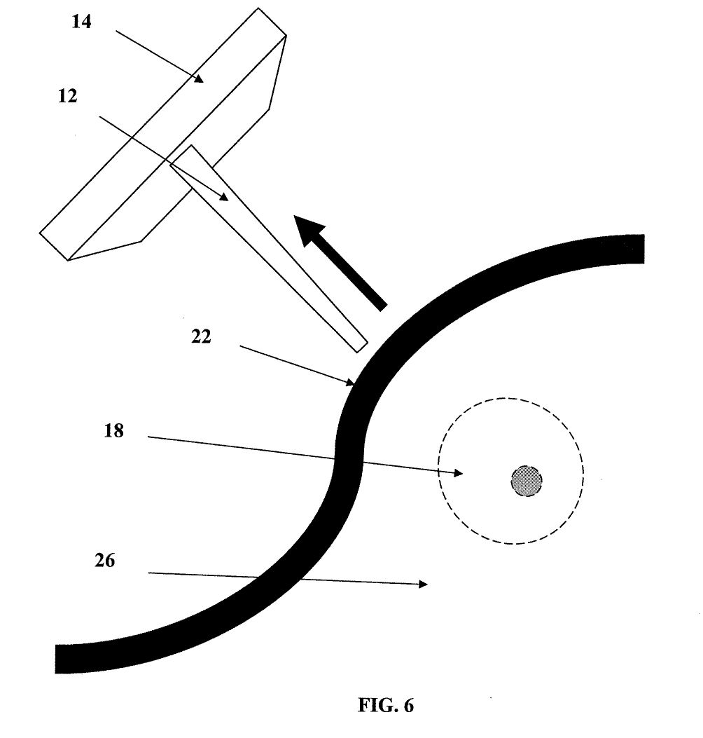

An exemplary delivery procedure is depicted in FIGS. 4-6. In FIG. 4, the immobilized therapeutic cell 16 is inserted through the skin 22 by piercing a penetration site 24 with the rod 12 towards the target site 26. Referring to FIG. 5, the therapeutic cell 16 is released from the rod 12 within or proximate to the target site 26, such as by reversing the magnetic polarity or depolarizing the rod 12. Removal of the depolarized rod 12 from the skin 22 is depicted in FIG. 6.

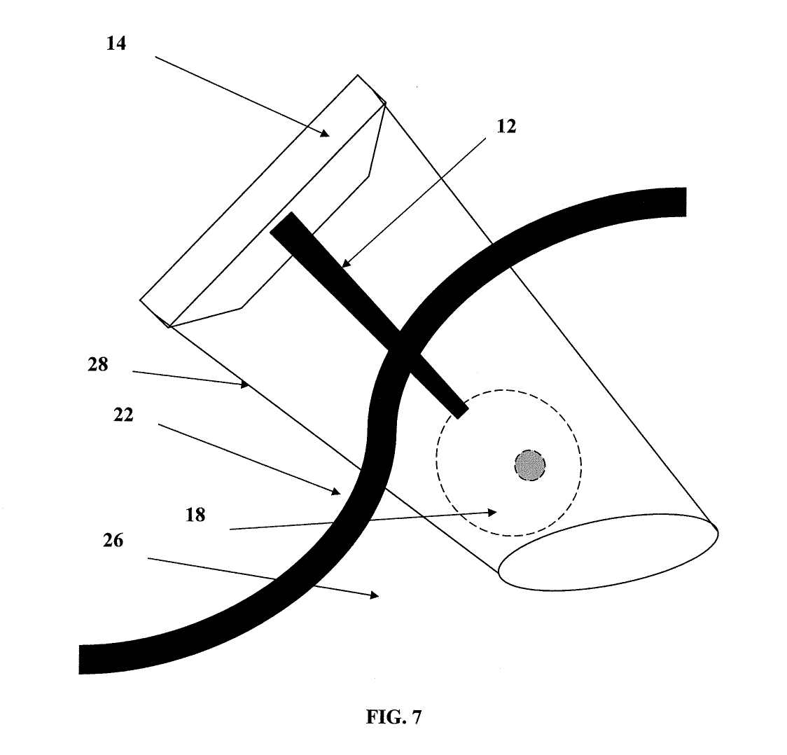

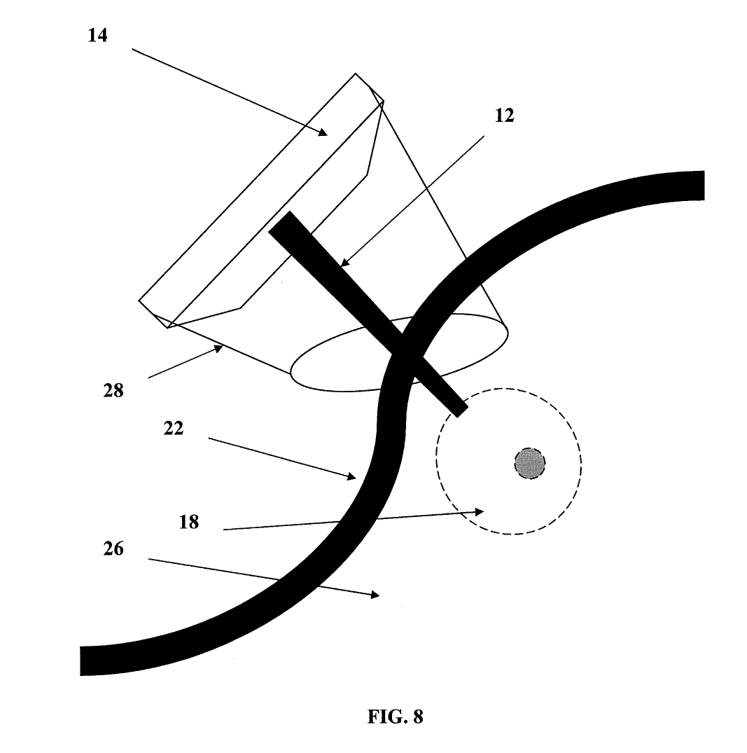

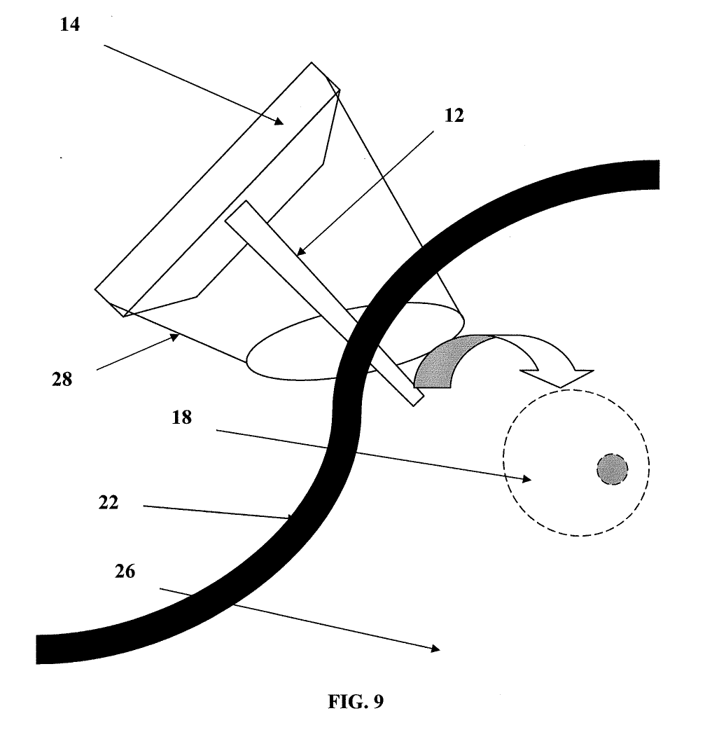

In FIGS. 7-9, a second exemplary delivery procedure is provided using the apparatus 10 having an optional sheath 28. In FIG. 7, the sheath 28 covers the rod 12 and therapeutic cell 16 during piercing of the penetration site 24. Referring to FIG. 8, once the therapeutic cell 16 is positioned within or proximate to the target site 26 the sheath 28 is retracted. FIG. 9 depicts the subsequent release of the therapeutic cell 16.

As summarized in FIGS. 2-9, the methods of the invention include collecting therapeutic cells 16 from the donor tissue followed by their delivery to a target site 26 in need of hair growth or restoration. The term “therapeutic cells” as used herein refers to cells having therapeutic benefit in the growth or production of hair. Therapeutic cells 16 may be found at or near to the hair follicle including the inner root sheath, outer root sheath, dermal coat, papilla, connective tissue papilla and the like. In some embodiments therapeutic cells 16 are present within the blood vessels supplying the hair follicle with cells and nutrients. As non-limiting examples, therapeutic cells 16 include hair follicle cells, stem cells and/or hair progenitor cells. In some embodiments the therapeutic cells are the dermal papilla cells. Stem cells found in the hair follicle may be the ultimate source of cells that generate the growth of new hairs. Stem cells may also be found in surrounding adipose tissue. In some embodiments, the therapeutic cells 16 are identifiable by the presence of a cell surface moiety or protein displayed at the cell surface. In some embodiments the cell surface moiety or surface protein is selected from the group of alpha-actin, Keratin 5/8, Keratin 15 and Nestin.

A variety of cell surface moieties, surface proteins or identifiable markers are associated at least in part with stem cells. Accordingly, the therapeutic cells 16 may include one or more surface moieties, surface proteins or expression markers selected from AA4, AA4.1, -ABCB1, ABCB5, ABCG2, AC133, ALDH, alkaline phosphatase, alpha6-integrin, antithrombin III (AT), asialo GM1, Bcl-2, beta1-integrin, bromodeoxyuridine, c-kit (CD117), c-Met, ClqR(p), CD105, CD133, -CD166, CD29, CD30, CD31, CD33, CD34, CD44, CD56, CD73, CD9, CD90, CDCP1, Circulating anticoagulants protein C (PC), CK15, CK19, CLV3, cyclic CMP, Cytokeratin (CK) 15, cytokeratin (CK) 19, ECMA-7, EDR1, EEC, FGF-4, Flk-2, Flk1(+)-Flt3/Flk2, FMS (CD115), FORSE-1, G alpha16, GDF3, GFPM, Gli2, Gli3, glial fibrillary acidic protein, glycoprotein IB, GSTA1, HAS2 gene expression, Her5, hMYADM, HSA, hsp25, Id2, IL-3Ralpha, Integrins, interleukin-3 receptor alpha chain, K19, KDR, keratin 15, Keratin 19 (K19), L-selectin (CD62L), Lamin A/C, Lewis X antigen (Le(X)), LeX, LgrS, Lrp4, MCM2, MCSP, MRP4, Msi-1, Musashi, Musashi-1, Mutant BCRP, Nestin, neurofilament microtubule-associated protein 2, neuron-glial antigen 2 (NG2), notch 1, nrp-1, Nucleostemin, OC.3, OCT4, OST-PTP, P-gp/MDR1, p21, p63, p75, PCLP, PCNA, PECAM, PgP-1, phosphorylating-p38, Podocalyxin, procalcitonin (PCT), PSCs, pSV2gpt, PTPRC, purified LRC, RC1 antigen, Rex-1, Sca-1, SCF, Side Population (SP), SOX10, SOX2, SOX9, SP phenotype, SSEA-1, SSEA-3, SSEA-4, Stat3, StatS, Stella, Stra8, Stro-1, Tartrate-resistant acid phosphatase (TRAcP), TdT, Thrombomucin, Thy-1, Tra-1-60, TWIST1, VEGFR-2, vimentin, X-Smoothened, XKrk1 and Zac1.

Most often the donor tissue will be taken from the same subject to be treated, albeit preferably at a region of relative higher density of hair than the target site 26. Thus preferably the donor tissue and target site 26 are autologous. However, in other embodiments the donor tissue and target site 26 are allogeneic, such as both human, and in still other embodiments the donor tissue and target site 26 are xenogeneic or belonging to different species. Most preferably the donor tissue includes removal or excision of tissue that includes at least an entire hair follicle, which may also include surrounding cells, the conjunctive tissue, arrector pili muscle and the like. Since the tissue is ultimately treated and harvested for desired therapeutic cells 16, the removal of the tissue does not require exact precision. In some embodiments adipose tissue is also collected. The collection of adipose tissue may be desired due to the presence of adipose tissue-derived stem cells.

Once excised, therapeutic cells 16 are harvested or collected from the donor tissue in part using an appropriate treatment protocol. Therapeutic cells 16 collected from a single hair follicle can be implanted at multiple target sites 26. Accordingly, the method is not limited to the transplantation of a single hair follicle to a new single location. In some embodiments the donor tissue is teased mechanically or morcellized in culture media with surgical tools to release therapeutic cells 16 into the surrounding media. In another approach the tissue is treated chemically to breakdown the epidermal and dermal tissue thereby releasing therapeutic cells 16 into a cocktail of cells or mixture of various cell types. In still another approach the tissue is enzymatically treated with an enzyme to release therapeutic cells 16 from the donor tissue.

Once released from donor tissue, cell populations may be assessed using techniques known in the art, such as cell-based ELISA assays, flow cytometry and the like. Cells may be cultured to propagate desired populations and the like as known in the tissue culture and cell biology arts. In some embodiments the therapeutic cells 16 are cultured to further expand one or more cell type or population. In other embodiments, cells 16 are genetically manipulated to deliver gene therapy, such as by incorporating sense or antisense DNA into the nucleus 18. For instance, therapeutic cells 16 may be genetically transformed, transfected, constructed or induced to express Fibroblast Growth Factor, Epidermal Growth Factor, Epidermal Growth Factor Receptor Inhibitors, Beta-Katenin and/or Angiogenin. In other embodiments, precursor cells are differentiated in culture to cells of desired lineage. Outer root sheath progenitor cells in the follicle have been shown to express Nestin; however, Nestin expression is lost during differentiation. Li et al., PNAS 100(17):9958-61 (2003). By monitoring the expression of markers associated with stem cells or differentiate cells, stem cells can be demonstrated to differentiate into cells such as dermal papilla cells, dermal sheath cells, afollicular epidermal cells, hair follicle bulge cells, outer-root sheath cells, and basal cells of the sebaceous glands, hair follicle cells and the like. Such stem cells or progenitor cells may be hair follicle stem cells, hair follicle progenitor cells, cells expressing Nestin and/or Keratin 5/8 and/or Keratin 15, epidermal stem cells, adipose derived stem cells. Further, differentiation can be further accomplished through the use of ADSC, marrow stem cells, mesenchymal stem cells, placental stem cells, peripherally circulating hematogenous stem cells, or de-differentiated pluripotent stem cells, induced pluripotent stem cells (iPS or iPSC) for hair transplants.

Therapeutic cells 16 are collected from cell cocktails or mixed cultures through specific binding interactions. Preferably the therapeutic cells 16 are exposed to magnetically sensitive antibodies 20 that bind cell surface moieties on the therapeutic cells 16. As used herein the term “antibody” or “antibodies” refers to immunoglobulins or fragments thereof, which includes a whole antibody, Fab, F(ab)′2, kappa fragment, lambda fragment and the like. The term “antibody” is intended to encompass, IgA, IgD, IgE and IgY, although IgM and IgG are typically preferred. Antibodies may be monoclonal or polyclonal and can be generated using a variety of cell lines, hosts and the like. The generation of antibodies is well known in the art as well as screening methods to identify specificity for a desired cell type. The skilled artisan will recognize that while the use of magnetically sensitive antibodies 20 is preferred a complementary binding protein having suitable affinity for a cell surface moiety may also be used.

By “magnetically sensitive antibody” it is meant that the antibody 20 is attracted to a magnetic source. Antibodies may be made magnetically sensitive by chemically conjugating metallic elements such iron, nickel or mixtures containing magnetic particles to the antibody. Some metals may propose a health risk in humans and thus may not be particularly preferred. Accordingly, compounds such as metal oxides have been developed which may prove safer. In some embodiments an iron oxide nanoparticle is used. Though ferromagnetic compounds may be used, preferably the magnetically sensitive antibody 20 is paramagnetic. A paramagnetic compound includes unpaired electrons, which when exposed to a magnetic field tend to align themselves in the same direction as the applied field. Once the field is removed the total magnetization drops to zero.

The magnetic features of the magnetically-sensitive antibodies may include nanoparticles, microparticles, beads and the like. A variety of magnetic beads have been conjugated to antibodies and thus the skilled artisan can select from a variety of protocols or kits as desired. For instance, it is well accepted that beads or particles having carboxyl groups can be reacted with amino groups of proteins through condensation reactions. It is also known to couple exposed amino groups using gluteraldehyde. However, such reactions can impair the binding between antibody and therapeutic cell 16. Though non-limiting in some instances the magnetic particle is conjugated to the Fc portion of a whole antibody. The term “conjugation” as used herein refers to direct chemical bonding between two compounds or moieties or the linking of two compounds through the use of an intermediary molecule, such as a molecule positioned between the magnetic particle and antibody that results in attachment, such as protein A domains to link Fc portions, bifunctional linkers and the like. Alternatively, antibodies may use adsorption without specific chemical linkage. Adsorption techniques are well known in the art, such as those that use styrene derived formulations. Proteins are known to bind nonspecifically to styrene. Thus, incorporating iron oxide into a styrene-derived polymer would adsorb antibodies and thus form magnetically-sensitive antibodies 20. Magnetically sensitive antibodies 20 bind therapeutic cells to form a cell-antibody complex. Each complex preferably includes a single cell or single cell type.

Immobilizing the cell-antibody complex can be performed by applying a magnetic field to the rod 12, which attracts magnetically sensitive antibodies 20 and thus therapeutic cells 16. An example of such an apparatus is summarized in FIG. 1, which depicts a magnetizable rod 12 attached to a base 14. In preferred embodiments the rod 12 is provided in an elongated pin configuration having a magnetizable tip and a shielded or magnetically insulated shaft, which focuses the magnetism at the tip. Generally only the tip of the rod 12 is exposed to the donor tissue and inserted into the target site 26 and thus it is generally preferable to insulate the majority of the rod 12. Methods of magnetically insulating or shielding portions are known in the art and may include administering an insulating or shielding coating, wrapping portions and the like.

Most preferably the tip of the rod 12 is magnetized electronically; however, the invention also encompasses embodiments where the tip is magnetized with a solid-state, predetermined magnetic polarity and strength without the need or use of electricity. Methods of generating electromagnetic fields are well known in the art. For instance, electromagnetic fields can be generated by moving electric current around a metal. Thus, movement of electric current around a magnetizable rod 12 can generate an electromagnet, which can attract magnetically sensitive antibodies 20 and thus the cell-antibody complex. Electronic circuitry or power sources, such as batteries, required to generate the electric magnet can be housed within the base 14.

In some embodiments a three dimension graft is generated by sequentially coating the rod 12 with layers of therapeutic cells 16. That is, a first or innermost layer of therapeutic cells 16 may have a different cell type than an outermost layer. Sequentially coating the rod 12 may be performed by exposing the magnetizable rod 12 to a first solution of a first cell-antibody complex followed by exposing the magnetizable rod 12 a second solution of a second cell-antibody complex. This procedure can be repeated as desired or as the magnetic forces of the rod 12 permit. For example, the rod 12 can be initially coated with a layer of keratinocyte stem cells or stem cells then coated with a layer of dermal papilla cells to form a 3 dimensional structure for implantation into the target site. An advantage of this approach is to offer a cocktail of therapeutic cells 16, possibly in a desired arrangement. The skilled artisan will appreciate that the rod 12 may be also be exposed to a wash solution, such as phosphate buffered saline, to wash away unbound cells or protein.

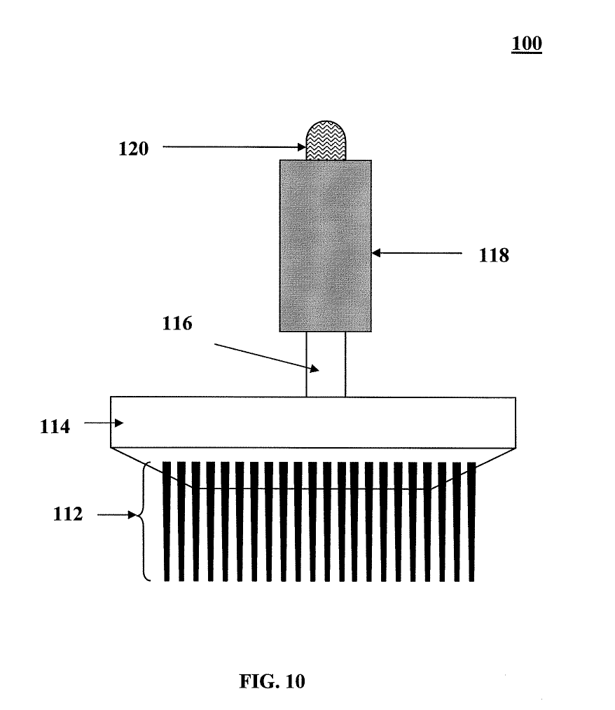

Turning to FIG. 10, a related apparatus 100 is provided, which includes a plurality of rods 112 attached to a base 114 to provide an array. This permits the immobilization of the cell-antibody complex across the entire array. Further, by providing an array that includes a plurality of rods 112 a variety of shapes and patterns can be constructed to simulate different hair patterns or densities. For instance, the base 114 may include a plurality of threaded apertures, each able to accept one of the plurality of rods 112. Threading the plurality of rods 112 in corresponding apertures may result in desired patterns or densities. Though non-limiting, the apparatus 100 can result in hair densities at about a minimum of 15-70 hairs per cm². Typically, rod 112 densities start out at a maximum density of 10×10⁶ per cm². Exemplary configurations of the array include ellipsoid, circular, ovoid, crescenteric, linear, curvilinear, square, triangular, rectangular, polygonal, convex, concave, sideburn-like, moustache-like, beard-like, male-pattern, female-pattern, tapered nape, straight nape and the like.

The apparatus 100 may also include a handle 116 with grip 118 to facilitate handling of the apparatus 100. The apparatus 100 may also include a button 120 or any suitable switching means, such as toggles, dials and the like to turn current on, off or change the polarity. Accordingly, the button 120 may selectively turn on and off the magnetic characteristics of the plurality of rods 112. In some embodiments the current can be controlled to increase or decrease the strength of the magnetic field. The button 120 or switching means may also selectively reverse the direction of current along the rod 112 and thus reverse the polarity of the magnetizable rod 112. Such an approach may be useful when wanting to repel the magnetically sensitive antibodies 20. In other configurations the rod 12 is depolarized thereby releasing the cell-antibody complex.

Once immobilized the therapeutic cells 16 may be delivered to the patient in balding areas or the like. Delivery is accomplished by piercing the skin 22 to reach a target site 26 followed by release of the cell-antibody complex. The target site 26 is the appropriate skin substrata, which is typically beneath the epidermal layer of the skin and at about the same depth as a traditional hair follicle. In some embodiments piercing of the skin 22 is performed directly using the rod 12, 112. In other embodiments piercing of the skin 22 is performed using a sheath 28 that optionally surrounds each rod 12, 112 and cell-antibody complex. Use of a sheath 28 may also function to protect from dislodging of the cell-antibody complex from the rod 12, 112 during piercing. The sheath 28 may be fixed in position or may be retractable as shown in FIG. 8. In other embodiments, the skin 22 is pierced to form cavities, followed by insertion of the rod 12, 112 into the cavity.

Once the rod 12, 112 is positioned at the target site 26 the rod 12, 112 is depolarized or reversed in polarity to release or repel the magnetically sensitive antibodies 20 from the rod 12, 112. The result is delivery of the therapeutic cells 16 to the target site 26. Since rod densities typically start out at a maximum density of 10×10⁶ per cm², therapeutic cell 16 concentrations and total number of cells 16 delivered per surface area may be modified to enhance engraftment and aesthetic appearance. The rod 12, 112 is then removed from the patient and repeated in additional areas as needed. Therapeutic cells 16 remain to propagate, assist with hair growth and the generation of new hair.

Since the apparatus 10, 100 and method are used preferably in humans, it is preferable to manufacture reagents and tools in an endotoxin free method. Such methods are well known in the art. Further, storage systems may also be provided such as those that store the rods 12, 112 in suitable medium, sterility and the like.

While the methods and apparatus have been demonstrated in specific configurations additional embodiments are also encompassed by the invention. For instance, in a first variation, therapeutic cells 16 are delivered together with a magnetically-sensitive therapeutic compound. The term “therapeutic compound” as used herein refers to a chemical that has a beneficial effect on the growth of hair, the prevention of cell death or the stimulation of therapeutic cells 16. The “therapeutic compound” may be a small molecule or drug. Compounds may be made “magnetically-sensitive” through conjugation or adsorption with appropriate nanoparticles, microparticles and the like. Accordingly, when releasing the magnetically sensitive antibody 20 the compound may be released simultaneously and at the same target site 26 as the therapeutic cells 16. A variety of therapeutic compounds may be provided, including a growth factor, a Fibroblast Growth Factor, Nestin, Epidermal Growth Factor, Epidermal Growth Factor Receptor Inhibitor, Human Growth Hormone, Insulin-like Growth Factors, insulin, Androgens, Angiogenin, Beta-Katenin, anti-sense RNA, RNAi, RNA, PNA, DNA, a protein and the like.

In some embodiments the therapeutic compound is an androgen. In other embodiments the compound affects the androgen receptor pathway, blocking or inhibiting the conversion of Testosterone into DHT and the like.

In some embodiments, the methods are performed together with a hair transplantation procedure. In such embodiments, the delivery of therapeutic cells 16 may enhance the growth of transplanted hair follicles.

### Example 1

### Obtaining Therapeutic Cells from Donor Tissue

Hair bearing skin specimens were incubated in the presence of dispase at 37 degrees celsius; for 2 h, the hair shafts with outer root sheath (ORS) embedded in the dermal sheath (DS) were extracted under dissecting microscope, and the ORS tissue were transferred to 4 well culture plates. The specimens were transected at the interface between the dermis and subcutaneous tissue. Sections of DS and DP (linked with and enclosed by DS) embedded in the adipose tissue were removed and incubated with collagenase at 37 deg C.; for 6-8 h, and the DP and DSCs were isolated by repeated low-speed centrifugation and cultured respectively on Petri dishes. The cultured ORS bulge cells were identified by immunohistochemistry with K19 antibody and DPCs and DSCs by immunohistochemistry with either alpha-actin, Nestin, keratin 5/8 or keratin 15 antibody.

What is claimed is:

1. A method for the treatment of hair loss comprising:
(a) excising donor tissue comprising therapeutic cells;
(b) treating the donor tissue to dissociate the therapeutic cells from the donor tissue;
(c) exposing the dissociated therapeutic cells to magnetically-sensitive antibodies capable of binding to a specific epitope of the therapeutic cells to form cell-antibody complexes;
(d) immobilizing the cell-antibody complexes to an electronically magnetizable rod;
(e) introducing the rod into a target site;
(f) releasing the cell-antibody complexes from the rod into the target site; and
(g) withdrawing the rod from the target site.

2. The method according to claim 1, wherein the donor tissue and the target site are selected from the group consisting of autologous, allogeneic and xenogeneic.

3. The method according to claim 1, wherein the donor tissue is skin tissue and the therapeutic cells are selected from the group consisting of hair follicle cells, stem cells, progenitor cells, marrow stem cells, mesenchymal stem cells, placental stem cells, peripherally circulating hematogenous stem cells, de-differentiated pluripotent stem cells, induced pluripotent stem cells (iPS or iPSC), fibroblasts, dermal papilla cells, and dermal sheath cells.

4. The method according to claim 1, wherein the donor tissue is adipose tissue and the therapeutic cells are adipose-derived stem cells.

5. The method according to claim 1, wherein the therapeutic cells comprise a series of different cell types; further wherein the method comprises sequentially coating the rod with cell-antibody complexes of the different cell types in different layers prior to introducing the rod into the target region to form a 3-dimensional graft.

6. The method according to claim 1, wherein the magnetically-sensitive antibodies are ferromagnetic.

7. The method according to claim 1, wherein the magnetically-sensitive antibodies are paramagnetic.

8. The method according to claim 1, wherein the step of immobilizing the cell-antibody complexes is performed by magnetically polarizing the rod.

9. The method according to claim 8, wherein the cell-antibody complexes are released from the rod into the target site by means of magnetically depolarizing the rod.

10. The method of claim 8, wherein the cell-antibody complexes are released from the rod into the target site by reversing the magnetic polarity of the rod, such that the cell-antibody complexes are repelled away from the rod.

11. The method according to claim 1, wherein the rod is introduced into the target site by piercing the skin with the rod, causing the cell-antibody complexes to be introduced under the surface of the skin.

12. The method according to claim 1, further comprising genetically manipulating the therapeutic cells prior to immobilizing the cell-antibody complexes on the rod to deliver gene therapy.

13. The method according to claim 1, further comprising immobilizing magnetically-sensitive small molecules or drugs on the rod prior to introducing the rod into the target site such that the small molecules or drugs are simultaneously released into the target site with the cell-antibody complexes.

14. The method according to claim 1, wherein the method is used for treating a human or a mammal.

15. A method for the treatment of hair loss, which comprises:
a) excising donor tissue comprising hair follicles from a subject;
b) treating the donor tissue to dissociate therapeutic cells from the hair follicles;
c) exposing the dissociated therapeutic cells to magnetically-sensitive antibodies capable of binding to a specific epitope of the therapeutic cells to form cell-antibody complexes;
d) immobilizing the cell-antibody complexes to an electronically magnetizable rod;
e) introducing the rod into a target site of the subject requiring hair growth;
f) releasing the cell-antibody complexes from the rod into the target site; and
g) withdrawing the rod from the target site.

16. An apparatus for use in the treatment of hair loss comprising a plurality of rods connected to a base and electronic circuitry, wherein each rod comprises a magnetically shielded shaft and an electronically magnetizable tip, further wherein the electronic circuitry adjusts the magnetic field strength and polarity of the plurality of magnetic tips.

17. The apparatus according to claim 16, wherein the number and pattern of rods is adjustable to simulate different hair densities and patterns.

18. The apparatus according to claim 16, further comprising a retractable sheath covering the rod tip to prevent damage to a cell-antibody complex immobilized thereon during penetration of skin tissue.

19. The apparatus according to claim 16, further comprising a handle connected to the base, wherein the handle comprises a button operably connected to the circuitry to adjust the magnetic field strength and/or polarity of the plurality of rods.

20. An apparatus for use in the treatment of hair loss comprising a plurality of rods connected to a base, wherein each rod comprises a magnetically shielded shaft and an electronically magnetizable tip, further wherein the plurality of rods comprise a solid-state, predetermined magnetic polarity and strength without electronic circuitry.
